(19) United States
(12) Patent Application Publication
(10) Pub. No.: US 2021/0346453 A1
(43) Pub. Date: Nov. 11, 2021

(54) COMPOSITIONS AND METHODS FOR TREATMENT OF COVID-19

(71) Applicant: Cognitive Clarity Inc., Edmonds, WA (US)

(72) Inventor: Alan D. Snow, Lynnwood, WA (US)

(73) Assignee: Cognitive Clarity Inc., Edmonds, WA (US)

(21) Appl. No.: 17/315,519

(22) Filed: May 10, 2021

Related U.S. Application Data
(60) Provisional application No. 63/022,655, filed on May 11, 2020.

(51) Int. Cl. A61K 36/74; A61K 45/06

(52) U.S. Cl. CPC A61K 36/74; A61K 9/48; A61K 45/06

(57) ABSTRACT

The present disclosure relates generally to methods for treating coronavirus infections (e.g., SARS-CoV-2 infection or COVID-19) in a subject in need thereof comprising administering to the subject a therapeutically effective amount of an Uncaria tomentosa extract.

### CROSS-REFERENCE TO RELATED APPLICATIONS

This application claims the benefit of and priority to U.S. Provisional Patent Application No. 63/022,655, filed May 11, 2020, the entire contents of which are incorporated herein by reference.

### TECHNICAL FIELD

The present technology relates generally to compositions comprising an Uncaria tomentosa extract, and methods of using the same to treat a coronavirus infection (e.g., COVID-19).

### BACKGROUND

The following description of the background of the present technology is provided simply as an aid in understanding the present technology and is not admitted to describe or constitute prior art to the present technology.

Coronaviruses are a group of enveloped viruses with positive strand large RNA genomes, ranging from 27-32 kilobases. Severe acute respiratory syndrome coronavirus (SARS-CoV) and Middle East respiratory syndrome coronavirus (MERS-CoV) are zoonotic coronaviruses that have caused regional and global outbreaks with mortality rate of 10% and 35%, respectively.

Coronavirus disease 2019 (COVID-19) (also referred to as novel coronavirus pneumonia or 2019-nCoV acute respiratory disease) is an infectious disease caused by the virus severe respiratory syndrome coronavirus 2 (SARS-CoV-2) (also referred to as novel coronavirus 2019, or 2019-nCoV). The disease was first identified in December 2019 and spread globally, causing a pandemic. COVID-19 is especially threatening to public health. The virus is highly contagious, and studies currently indicate that it can be spread by asymptomatic carriers or by those who are pre-symptomatic. There is not yet a specific approved antiviral or prophylaxis treatment for COVID-19 and accordingly, there is a pressing need for safe and effective treatments for coronavirus infections and for treatment of SARS-CoV-2 infection (e.g., COVID-19).

### SUMMARY OF THE PRESENT TECHNOLOGY

In one aspect, the present disclosure provides a method for treating coronavirus infection in a subject in need thereof comprising administering to the subject a composition comprising a therapeutically effective amount of an Uncaria tomentosa extract. In some embodiments, the composition comprises from about 100 mg to about 500 mg of an Uncaria tomentosa extract. Additionally or alternatively, in certain embodiments, the composition comprises from about 10% to about 40% w/w Uncaria tomentosa extract. The composition may be formulated as a pill, tablet, caplet, soft or hard gelatin capsule, lozenge, sachet, cachet, vegicap, liquid drop, elixir, suspension, emulsion, solution, beverage preparation, cold or hot tea beverage, syrup, tea bag, aerosol, suppository, sterile injectable solution, or sterile packaged powder. In certain embodiments, the composition is formulated as a capsule. In a further embodiment, the capsule is from about 200 mg to about 1000 mg.

Additionally or alternatively, in some embodiments of the methods disclosed herein, the subject is at risk for contracting a coronavirus infection, or has contracted a coronavirus infection. Examples of coronavirus infection include, but are not limited to, MERS-CoV infection (e.g., Middle East Respiratory Syndrome), SARS-CoV infection (e.g., Severe Acute Respiratory Syndrome), and SARS-CoV-2 infection (e.g., COVID-19). In certain embodiments, the coronavirus infection is SARS-CoV-2 infection (e.g., COVID-19). Symptoms of coronavirus infection may include one or more of coughing, dizziness, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness, confusion, blueish face or lips, coughing up blood, decreased white blood cell count, and palpitations. Coronavirus infection may cause one or more complications selected from the group consisting of sinusitis, otitis media, pneumonia, acute respiratory distress syndrome, disseminated intravascular coagulation, pericarditis, pulmonary fibrosis, viral sepsis, and kidney failure.

Additionally or alternatively, in some embodiments of the methods disclosed herein, the composition is administered orally, topically, intranasally, systemically, intravenously, subcutaneously, intraperitoneally, intradermally, intraocularly, iontophoretically, transmucosally, intramuscularly, intrathecally, intracerebrally, intranodally, intrapleurally, or intracerebroventricularly. Additionally or alternatively, in some embodiments, the method further comprises separately, sequentially or simultaneously administering one or more additional therapeutic agents to the subject. Examples of additional therapeutic agents include, but are not limited to, baricitinib, Vitamin C, Vitamin D, zinc, hesperetinj, melatonin, an anticoagulant, oxygen therapy, antivirals (Lopinavir, Ritonavir, Ribavirin, Favipiravir (T-705), remdesivir, oseltamivir, chloroquine, hydroxychloroquine, merimepodib, and Interferon), dexamethasone, prednisone, methylprednisolone, hydrocortisone, anti-inflammatory therapy, convalescent plasma therapy, bamlanivimab, etesevimab, casirivimab, imdevimab, and combinations thereof.

### BRIEF DESCRIPTION OF THE DRAWINGS

FIGS. 1A-1D show the chemical structures of quinoline drugs hydroxychloroquine and chloroquine in comparison to the alkaloid quinine (derived from Chinchona bark), and oxindole alkaloids derived from cat's claw (Uncaria tomentosa) bark. FIG. 1A shows the chemical structure of the synthetic quinoline drug hydroxychloroquine. FIG. 1B shows the chemical structure of synthetic chloroquine. FIG. 1C shows the chemical structure of the alkaloid quinine isolated from Chinchona bark powder. FIG. 1D shows chemical structures of the major oxindole alkaloids found in cat's claw (Uncaria tomentosa) bark powder.

FIGS. 2A-2F show the link between hydroxychloroquine, quinine and cat's claw lies in the bark. FIG. 2A shows that the alkaloid quinine and anti-malarial drug is derived from the Chinchona tree (family Rubiaceae) in South America and is the national tree of Peru. FIG. 2B shows Chinchona red bark bundles derived from the Chinchona tree to which quinine is isolated. FIG. 2C shows Chinchona bark powder. FIG. 2D shows that the woody vine cat's claw (Uncaria tomentosa) grows in the Amazon rain forest and has distinctive claw-like thorns which project from the base of its leaves. Uncaria tomentosa belongs to the same family Rubiaceae as the Chinchona tree from which the anti-malarial drug quinine is derived. FIG. 2E shows Cat's claw (also known as Uňa de Gato) bark bundles sold in the Peruvian marketplace. FIG. 2F shows that Cat's claw (Uncaria tomentosa) bark powder contains oxindole and pentacyclic alkaloids and polyphenols with anti-viral and anti-inflammatory properties.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, or topically. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, “comprising” shall mean that the methods and compositions include the recited elements, but not exclude others. “Consisting essentially of” when used to define methods and compositions, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of the present technology or process steps to produce a composition or achieve an intended result. Embodiments defined by each of these transitional terms and phrases are within the scope of the present technology.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject”, “patient”, or “individual” can be an individual organism, a vertebrate, a mammal, or a human. In some embodiments, the subject, patient or individual is a human.

As used herein, the term “therapeutic agent” is intended to mean a compound that, when present in an effective amount, produces a desired therapeutic effect on a subject in need thereof. “Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Coronaviruses and Coronavirus Infections

Coronaviruses are enveloped positive sense RNA viruses ranging from 60 nm to 140 nm in diameter with spike-like projections on its surface giving it a crown like appearance under the electron microscope; hence the name coronavirus. Four corona viruses namely HKU1, NL63, 229E and OC43 have been in circulation in humans, and generally cause mild respiratory disease. Severe acute respiratory syndrome coronavirus (SARS-CoV) and Middle East respiratory syndrome coronavirus (MERS-CoV) are zoonotic coronaviruses that have caused regional and global outbreaks with mortality rate of 10% and 35%, respectively (Chan-Yeung M, Xu R H, Respirology 8: S9-14 (2003); www.who.int/emergencies/mers-cov/en/).

Coronavirus disease 2019 (COVID-19) (also referred to as novel coronavirus pneumonia or 2019-nCoV acute respiratory disease) is an infectious disease caused by the virus severe respiratory syndrome coronavirus 2 (SARS-CoV-2) (also referred to as novel coronavirus 2019, or 2019-nCoV). SARS-CoV-2 infection (e.g., COVID-19) and its corona virus has led to a worldwide pandemic. SARS-CoV-2 infection (e.g., COVID-19) is especially threatening to public health. The virus is highly contagious, and studies currently indicate that it can be spread by asymptomatic carriers or by those who are pre-symptomatic. Likewise, the early stage of the disease is slow-progressing enough that carriers do not often realize they are infected, leading them to expose numerous others to the virus. The combination of COVID-19′s ease of transmission, its high rate of hospitalization of victims, and its death rate make the virus a substantial public health risk, especially for countries without a healthcare system equipped to provide supportive care to pandemic-level numbers of patients. There is not yet a specific antiviral or prophylaxis treatment for SARS-CoV-2 infection (e.g., COVID-19).

Genetic variants of SARS-CoV-2 have been emerging and circulating around the world throughout the COVID-19 pandemic. Table 1 provides a non-exhaustive list of SARS-CoV-2 variants identified in the United States (updated May 5, 2021. https://www.cdc.gov/coronavirus/2019-ncov/cases-updates/variant-surveillance/variant-info.html):

TABLE 1

| ID^{a} | Spike Protein Substitutions | First Detected |
|---|---|---|
| B.1.526 | Spike: (L5F*), T95I, D253G, (S477N*), | United States (New York)- |
| | (E484K*), D614G, (A701V*) | November 2020 |
| B.1.526.1 | Spike: D80G, Δ144, F157S, L452R, | United States (New York)- |
| | D614G, (T791I*), (T859N*), D950H | October 2020 |
| B.1.525 | Spike: A67V, Δ69/70, Δ144, E484K, | United Kingdom/Nigeria- |
| | D614G, Q677H, F888L | December 2020 |
| P.2 | Spike: E484K, (F565L*), D614G, V1176F | Brazil-April 2020 |
| B.1.617 | Spike: L452R, E484Q, D614G | India-February 2021 |
| B.1.617.1 | Spike: (T95I), G142D, E154K, L452R, | India-December 2020 |
| | E484Q, D614G, P681R, Q1071H | |
| B.1.617.2 | Spike: T19R, (G142D), Δ156, Δ157, | India-December 2020 |
| | R158G, L452R, T478K, D614G, P681R, | |
| | D950N | |
| B.1.617.3 | Spike: T19R, G142D, L452R, E484Q, | India-October 2020 |
| | D614G, P681R, D950N | |
| B.1.1.7 | Δ69/70, Δ144, (E484K*), (5494P*), | United Kingdom |
| | N501Y, A570D, D614G, P681H, T716I, | |
| | S982A, D1118H (K1191N*) | |
| P.1 | L18F, T20N, P26S, D138Y, R190S, | Japan/Brazil |
| | K417T, E484K, N501Y, D614G, H655Y, | |
| | T1027I | |
| B.1.351 | D80A, D215G, Δ241/242/243, K417N, | South Africa |
| | E484K, N501Y, D614G, A701V | |
| B.1.427 | L452R, D614G | United States-(California) |
| B.1.429 | S13I, W152C, L452R, D614G | United States-(California) |

*detected in some but not all sequences
^{a}Phylogenetic Assignment of Named Global Outbreak (PANGO) Lineages is software tool developed by members of the Rambaut Lab. The associated web application was developed by the Centre for Genomic Pathogen Surveillance in South Cambridgeshire and is intended to implement the dynamic nomenclature of SARS-CoV-2 lineages, known as the PANGO nomenclature.

The pathological hallmarks of the disease include prominent pneumonia lesions in the lungs, mainly manifested by alveolar exudate inflammation and interstitial inflammation, alveolar epithelial cell proliferation and hyaline membrane formation. The alveolar structures are usually damaged to varying degrees, with small amounts of serous fluid and fibrinous exudates observed in the alveolar cavities. Alveolar septal vascular congestion, edema, widening, various amounts of mononuclear and lymphocyte infiltration; focal bleeding in the lung tissue, partial alveolar exudation mechanism and pulmonary interstitial fibrosis are observed. Coronavirus particles are usually seen in the cytoplasm of the airway mucosa epithelium and type II alveolar epithelial cells under the electron microscope.

Symptoms of SARS-CoV-2 infection (e.g., COVID-19) include fever, dizziness, cough, shortness of breath, fatigue, headache, loss of smell, nasal congestion, sore throat, coughing up sputum, pain in muscles or joints, chills, nausea, vomiting, and diarrhea. In severe cases, symptoms can include difficulty waking, confusion, blueish face or lips, coughing up blood, decreased white blood cell count, and kidney failure. Complications can include pneumonia, viral sepsis, acute respiratory distress syndrome, and kidney failure. The progression of the diseases is associated with extreme rise in inflammatory cytokines including IL2, IL7, IL10, GCSF, IP10, MCP1, MIP1A, and TNFα (Chen N, et al., Lancet 395: 507-513 (2020)). The median time from onset of symptoms to dyspnea is 5 days, hospitalization 7 days and acute respiratory distress syndrome (ARDS) 8 days. The need for intensive care admission was in 25-30% of affected patients in published series. Recovery started in the 2nd or 3rd weeks. The median duration of hospital stay in those who recovered was 10 days. Adverse outcomes and death are more common in the elderly and those with underlying co-morbidities (50-75% of fatal cases). Fatality rate in hospitalized adult patients ranged from 4 to 11%. The overall case fatality rate is estimated to range between 2 and 3% (www.worldometers.info/coronavirus/).

### Screening and Diagnosis of Coronavirus Infections

The complementary tests used in the diagnosis of coronavirus infection (e.g., SARS-CoV-2 infection or COVID-19) can be divided into tests for etiological diagnosis and support tests, which help in the diagnosis or indicate the risk or presence of complications. Tests for etiological diagnosis. Tests for etiological diagnosis may be direct, identifying genetic material of coronavirus (e.g., SARS-CoV-2) or indirect, determining the humoral immune response to coronavirus (e.g., SARS-CoV-2). The most commonly used method for identifying genetic material from SARS-CoV-2 is real-time polymerase chain reaction (RT-PCR). Serological tests identify the presence of humoral response to coronavirus (e.g., SARS-CoV-2). Antibodies of IgA, IgM, and IgG isotypes specific to different virus proteins are detected by enzyme-linked immunosorbent assay (ELISA) or chemiluminescence immunoassays (CLIA).

Although detection of viral RNA remains the gold standard, false-negative results are not uncommon. Clinical diagnosis is generally based on exposure history, clinical symptoms, result of blood and biochemical tests, and findings on chest tomography (CT) which typically consists of ground-glass opacities (GGOs) or bilateral consolidations in multiple lobular and sub-segmental areas.

Support tests. These are laboratory or imaging tests that demonstrate characteristic manifestations of coronavirus infection (e.g., SARS-CoV-2 infection), its complications, and/or risk factors for complications. These support tests are well-known to a person skilled in the art. Although the findings in these tests are not specific to coronavirus infection (e.g., SARS-CoV-2 infection), given a compatible clinical picture and/or the presence of confirmed or possible history of contact, they may help in the diagnosis. Non-limiting examples are the support tests used in clinic to aid diagnosis of SARS-CoV-2 infection (e.g., COVID-19), which are provided below.

Complete blood count—lymphopenia, eosinopenia, and neutrophil/lymphocyte ratio≥3.13 are related to greater severity and worse prognosis.

Thrombocytopenia is related to a higher risk of myocardial damage and a worse prognosis.

Lymphopenia results from a multifactorial mechanism that includes the cytopathic effect of the virus, induction of apoptosis, Interleukin-1-mediated pyroptosis, and bone marrow suppression by inflammatory cytokines.

High values of C-reactive protein (CRP), ferritin, D-dimer, procalcitonin, lactic dehydrogenesis (DHL), prothrombin time, activated partial thromboplastin time, amyloid serum protein A, creatine kinase (CK), glutamic-pyruvic transaminase (SGPT), urea, and creatinine are risk factors for more severe disease, thromboembolic complications, myocardial damage, and/or worse prognosis.

Immunological markers that may also represent risk factors for greater severity and/or worse prognosis are: decreased values of CD4+T and CD8+ lymphocytes, and NK cells and increased values of IL-6, IL-8, IL-10, IFN-γ, TNF-IL-2R, TNF-α, GM-CSF, and IL-1 β.

Plain chest X-rays may evidence sparse bilateral consolidations accompanied by ground glass opacities, peripheral/subpleural images, predominantly in the lower lobes.

Computed tomography of the chest presents greater sensitivity and reveals multifocal, bilateral, peripheral/subpleural ground glass opacities, generally affecting the posterior portions of the lower lobes, with or without associated consolidations. Children have a similar presentation to that found in adults, albeit with a milder involvement. The halo sign, described as a consolidation area involved by ground glass opacities, was identified in 50% of the children. An inverted halo sign, in which areas of ground glass opacities are surrounded by condensation halo, has also been described.

Pulmonary ultrasonography has good sensitivity; the typical findings are B-lines, consolidations and pleural thickening.

### Existing Treatments of Coronavirus Infection

Several safe and highly effective vaccines against SARS-CoV-2 infection (e.g., COVID-19) have been given emergency use authorization in US, including Pfizer-BioNTech or Moderna COVID-19 vaccine, and Johnson & Johnson's Janssen COVID-19 vaccine.

There are some potential treatments for SARS-CoV-2 infection (e.g., COVID-19), most of which originate from previous therapeutic approaches for MERS and SARS. However, all of them are still under development or investigation and the treatment guidelines for COVID-19 vary between countries. Examples of such therapies include but are not limited to oxygen therapy, antivirals (Lopinavir, Ritonavir, Ribavirin, Favipiravir (T-705), remdesivir, oseltamivir, chloroquine, hydroxychloroquine, merimepodib, and Interferon), dexamethasone, prednisone, methylprednisolone, hydrocortisone, anti-inflammatory therapy, convalescent plasma therapy, bamlanivimab, casirivimab and imdevimab.

General treatments. General treatments include supportive treatments and rest. These actions regulate sufficient daily energy intake and monitor the vital signs such as oxygen saturation, respiratory rate, and heart rate. As for patients with mild COVID-19, symptomatic treatment such as antipyretics for fever and pain, adequate nutrition, and rehydration are recommended by the World-Health Organization (WHO). Beyond that, some treatments may be used for people who have been hospitalized with COVID-19, and other medications are indicated as being able to curb the progression of COVID-19 in people who are not hospitalized but who are at risk for developing severe illness.

Anti-inflammatory agents. Dexamethasone and other corticosteroids (prednisone, methylprednisolone) are potent anti-inflammatory drugs, and have been indicated as beneficial to certain patients with hospitalized with severe COVID-19.

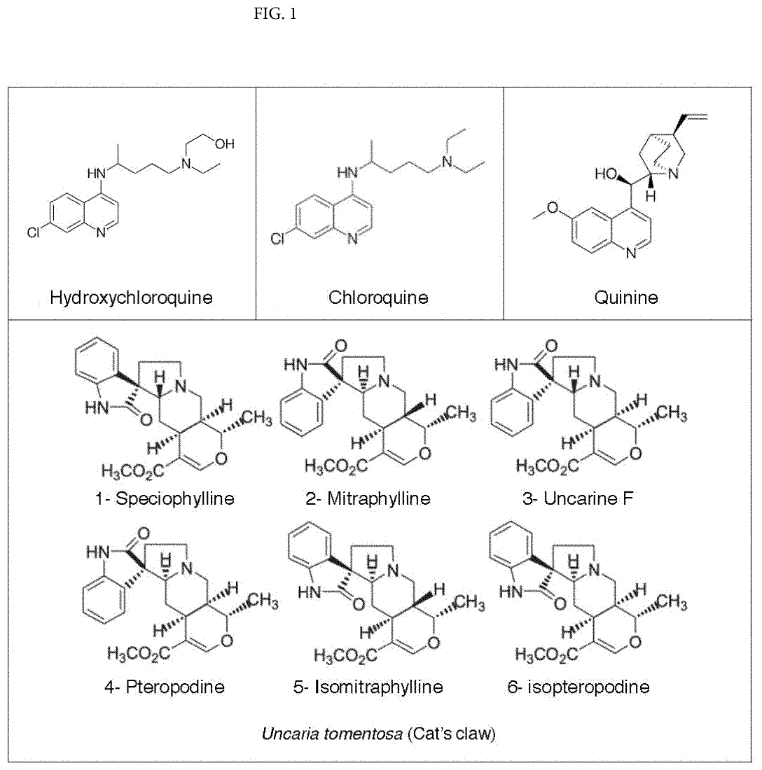

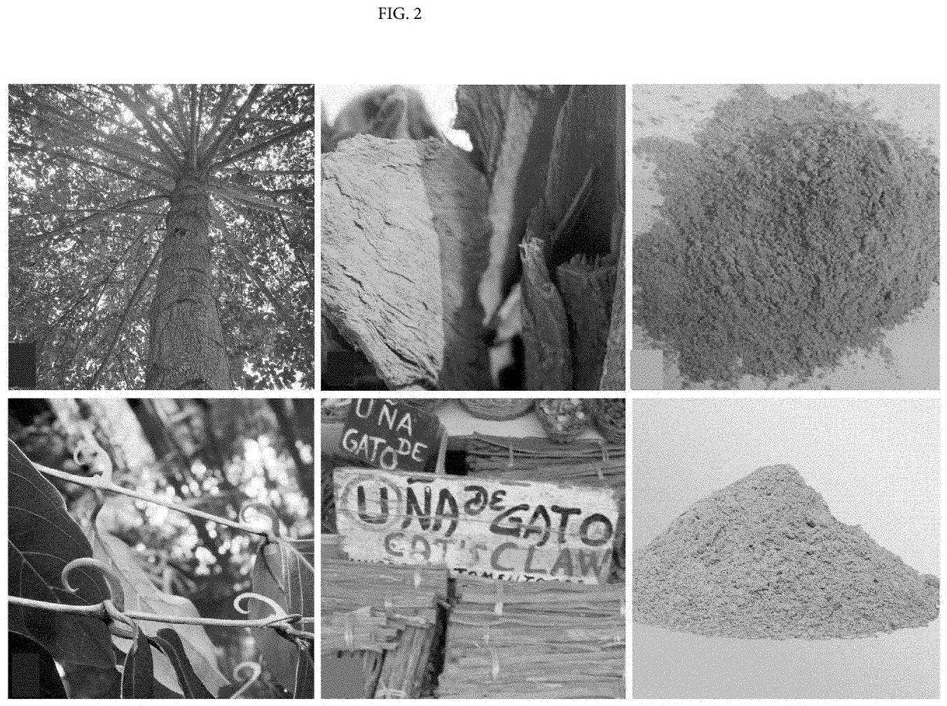

Antiviral agents. Chloroquine (FIG. 1B) and Hydroxychloroquine (FIG. 1A) (both are 4-aminoquinoline derivatives of Quinine (FIG. 1C), which is extracted from the bark of a Cinchona tree native to Peru (FIGS. 2A-2C)), IFN-I, and Remdesivir have been proposed to treat Covid-19.

Combination Therapy. Bamlanivimab alone and the combination of casirivimab and imdevimab were both found to significantly reduce the risk of being hospitalized or visiting the Emergency Room within 28 days after treatment, compared to placebo. The bamlanivimab/etesevimab combination was found to significantly reduce the risk of hospitalization or death within 29 days of treatment, compared to placebo. These treatments are not authorized for hospitalized COVID-19 patients or those receiving oxygen therapy. They have not shown to benefit these patients and could lead to worse outcomes in these patients.

Monoclonal antibodies. The three monoclonal antibody treatments that have received emergency use authorization in the USA are bamlanivimab, made by Eli Lilly; a combination of casirivimab and imdevimab, made by Regeneron; and a combination of bamlanivimab and etesevimab, made by Eli Lilly. These treatments must be given intravenously in a clinic or hospital. The three monoclonal antibody treatments were tested in separate clinical trials. Bamlanivimab alone and the combination of casirivimab and imdevimab were both found to significantly reduce the risk of being hospitalized or visiting the ER within 28 days after treatment, compared to placebo. The bamlanivimab/etesevimab combination was found to significantly reduce the risk of hospitalization or death within 29 days of treatment, compared to placebo. These treatments are not authorized for hospitalized COVID-19 patients or those receiving oxygen therapy. They have not shown to benefit these patients and could lead to worse outcomes in these patients.

Convalescent plasma. In August 2020, an emergency use authorization was issued for convalescent plasma (i.e., plasma from recovered Covid-19 patients) in patients hospitalized with COVID-19 in US. However, in a clinic trial including 1,060 patients with COVID-19 who received either convalescent plasma, a placebo, or standard treatment, treatment with convalescent plasma compared with control was not associated with improved survival or other positive clinical outcomes (Perrine J., et al., JAMA 325: 1185-1195 (2021)). Accordingly, the current treatments and combinations thereof do not appear to be optimal for the treatment of COVID-19.

### Compositions of the Present Technology

The present technology provides compositions comprising a therapeutically effective amount of Uncaria tomentosa extracts and methods useful in the treatment of coronavirus infection in a subject.

There are about 34 species of Uncaria, with Uncaria tomentosa being the most common species. The plant Uncaria tomentosa, also known as “Uña de Gato” (in Spanish) or “Cat's claw” (in English) refers to a woody vine which grows within the Amazon rain forest. Other names for Cat's claw also include Paraguayo, Garabato, Garbato casha, Tambor huasca, Una de gavilan, Hawk's claw, Nail of Cat, and Nail of Cat Schuler. This slow-growing vine takes 20 years to reach maturity and can grow over 100 feet in length as it attaches and wraps itself around the native trees. It is found abundantly in the foothills, at elevations of two to eight thousand feet. The vine is referred to as “cat's claw” because of its distinctive curved claw-like thorns that project from the base of its leaves (FIG. 2D). The bark bundles from the base of the tree where the medicinal properties lie are sold in the Peruvian marketplace as cat's claw or Una de Gato (FIG. 2E). The cat's claw bark is harvested and extracted for medicinal purposes (FIG. 2F). It is found abundantly in the foothills in the Amazon rain forest at elevations of 2,000 to 8,000 feet.

The cat's claw bark contains important alkaloids and polyphenols shown to exhibit potent anti-inflammatory activity. Cat's claw is found in nature in two different chemotypes producing different alkaloidal constituents. Pentacyclic oxindoles are found in one type, where the tetracyclic alkaloids are present in the second type. Oxindole and pentacyclic alkaloids found in cat's claw include mitraphylline, pteropodine, isomitraphylline, rhynchophylline and isorhynchophylline (FIG. 1D). Besides the presence of alkaloids, Uncaria tomentosa has been found to contain other phytochemicals including quinic acid, quinovic acid glycosides, low molecular weight polyphenols, ursolic acid, oleanolic acid, beta-sitosterol, stigmasterol, campesterol, and the three polyhydroxylated triterpenes. In addition, rotundifoline, isorotundifoline, quinovic acid, flavanoids and courmarins have additionally been isolated and identified in Uncaria tomentosa. The inventors of the present disclosure discovered new and specific polyphenolic epicatechin-dimers (i.e., proanthocyanidins) in Uncaria tomentosa including epicatechin-4β-8-epicatechin (proanthocyanidin B2), catechin-4-α-8-epicatechin (proanthocyanidin B4), epicatechin-4β-8-epicatechin-4β-8-epicatechin (proanthocyanidin C1) and epiafzelechin-4β-8-epicatechin found to inhibit and reduce brain plaques and tangles in the aging brain (Snow et al., Scientific Rep. 9: 561 (2019)).

Uncaria tomentosa has been shown to possess potent anti-viral activity against the Dengue virus (Aquino et al., J. Nat. Prod. 52: 679-685 (1989); Lin et al., J. Trad. Compl. Med. 4: 24-35 (2014); Mackenzie et al., Nat. Med. 10: S98-S109 (2004); Hastead, Science 239: 476-481 (1988); Scott et al., J. Infect. Dis. 141: 1-6 (1980); Reis et al., Int. Immunopharm. 8: 468-476 (2008); Mello et al., Mem. Inst. Oswaldo Cruz. 112: 458-468 (2017)), herpes simplex (Caon et al., Food Chem. Tox. 66: 30-35 (2014); Williams, Alt. Med Rev. 6: 567-579 (2001); Hassan et al., J. Pharm. Pharmacol. 67: 1325-133 (2015)), Epstein-Barr (Williams, Alt. Med. Rev. 6: 567-579 (2001)) and HIV (Williams, Alt. Med. Rev. 6: 567-579 (2001)). Dengue fever is an endemic in tropical and subtropical regions in South America, Southeast Asia, Pacific Islands and the Americas. Dengue virus targets mainly mononuclear cells such as monocytes and there is also a marked “cytokine storm” (similar to COVID-19) with a marked increase of inflammatory cytokines including interleukin-1 and TNF-α. In human monocytes infected with Dengue virus-2, Uncaria tomentosa extract and its pentacyclic oxindole alkaloid enriched-fractions reduced DENV Virus-2 antigen in treated monocytes, and markedly reduced the inflammatory cytokines TNF-α and Interleukin-1 (Reis et al., Int. Immunopharm. 8: 468-476 (2008)). In another study, Uncaria guianensis (another popular species of Uncaria with similar constituents) reduced intracellular viral antigen and inhibited the secretion of viral non-structural protein, which is indicative of viral replication (Mello et al., Mem. Inst. Oswaldo Cruz. 112: 458-468 (2017)). Preparations of Uncaria tomentosa (quinovic acid glycosides and oxindole alkaloids) also demonstrated inhibition of viral attachment to the cell using herpes simplex viruses (Caon et al., Food Chem. Tox. 66: 30-35 (2014); Williams, Alt. Med. Rev. 6: 567-579 (2001); Hassan et al., J. Pharm. Pharmacol. 67: 1325-133 (2015)) with the polyphenols present in Uncaria tomentosa appearing to be responsible for the observed effects (Snow et al., Scientific Rep. 9: 561 (2019); Williams, Alt. Med. Rev. 6: 567-579 (2001)).

Uncaria tomentosa (cat's claw) also has potent anti-mutagenic activity and is an enhancer of DNA repair (Mammone et al., 2006 Phytother. Res. March 6. https://doi.org/10.1002/ptr.1827; Sheng et al., (2000) Ethnopharmacol. 69, 115-126; Sheng et al., (2001) Phytomed. 8, 275-282; Rizzi et al., (1993) J Ethnopharmacol. 38, 63-77). In a human volunteer study, there was a statistically significant increase in DNA repair when 250 mg and 350 mg tablets of Uncaria tomentosa were administered over an 8-week period (Sheng et al., 2001). Additionally, Uncaria tomentosa was deemed safe based on clinical symptoms, serum clinical chemistry, whole blood analysis and leukocyte differential counts.

Uncaria tomentosa (cat's claw) additionally has potent anti-inflammatory activity and is a potent enhancer of the immune system. It is also a potent inhibitor of TNF-α and interleukin-1 (Aguilar et al., Ethnopharmacol. 81: 271-276 (2002); Sandoval et al., Free Radic. Biol. Med. 29: 71-78 (2000)), and negates the activation of NF-κB (Allen-Hall et al., 2010; Sandoval-Chacon et al., 1998). Both the alkaloids and polyphenols (proanthocyanidins) present in Uncaria tomentosa appear to contain potent anti-inflammatory activity (Aquino et al., J. Natural Prod. 54: 453-459 (1991); Mur et al., J. Rheumatol. 29, 678-681 (2002); Snow et al., Scientific Rep. 9: 561 (2019)). Proanthocyanidin B2 (epicatechin-4β-8-epicatechin dimer) isolated from Uncaria tomentosa can remarkedly reduce inflammation in brain (astrocytosis and microgliosis) in plaque-producing transgenic mice by 69%-80.3% within 3 months of treatment. (Snow et al., Scientific Rep. 9: 561 (2019)).

In an ozone-induced lung inflammation animal model (which mimics some of the respiratory tract inflammation and “cytokine storm” as observed with COVID-19), increasing concentrations of Uncaria tomentosa significantly reduced lung inflammation in a dose-dependent manner immediately, and after 8 hours of treatment (Cisneros et al., J. Ethnopharmacol. 96: 355-364 (2005)). In this ozone-induced animal model, there was an accumulation of protein-rich fluid in the alveolar and brochiolar epithelium, with extensive capillary leakage (Cisneros et al., J. Ethnopharmacol. 96: 355-364 (2005); Kleeberger and Hudak, J. Appl. Physiol. 72: 670-676 (1992)). Uncaria tomentosa markedly and in a dose-dependent manner reduced the edema and inflammation in lungs, capillary leakage of protein rich serum into the airways (Kleeberger and Hudak, J. Appl. Physiol. 72: 670-676 (1992)). It was also suggested that oral administration of Uncaria tomentosa orally for 8 days prior to the O³ exposure would elicit a lung tissue protective effect (Cisneros et al., J. Ethnopharmacol. 96: 355-364 (2005). In a randomized double-blind human trial, an extract from the pentacyclic alkaloid-chemotype of Uncaria tomentosa was shown to be safe and improved joint pain in patients with rheumatoid arthritis taking hydroxychloroquine or sulfasalazine (Mur et al., J. Rheumatol. 29, 678-681 (2002)).

Without wishing to be bound by theory, it is believed that anti-viral and anti-inflammatory properties of Uncaria tomentosa extracts render them useful in methods for treating coronavirus infection in a subject in need thereof.

For the present disclosure, commercially available Uncaria tomentosa extracts may be obtained from various vendors. Examples of commercially available Uncaria tomentosa extracts include but are not limited to those marketed by Ziggy Health, Bulk Supplements, Brainchild Nutritionals, Swanson, Now Foods, Herb Pharm, Quicksilver Scientific, Mary Ruth Organics, Z natural Food, Gaia Herbs, Herbal Goodness, Puritan's pride, Hawaii Pharm, Source Naturals, Mountain Rose Herbs, Nature's Answer, Prescribed For Life, Pure Mountain Botanicals, Fresh Thyme, and EnzymeProcess. In some embodiments, the Uncaria tomentosa extract is PTI-00703®. PTI-00703® is derived from cat's claw bark found in the Amazon rainforest of Peruvian source. PTI-00703® cat's claw comprises a 70% ethanol/water (v/v) extract of Uncaria tomentosa bark powder that is filtered (to remove high molecular weight material) and finally concentrated by spray drying.

In some embodiments, the Uncaria tomentosa extract comprises a 10%-90% alcohol/water (v/v) extract of Uncaria tomentosa bark powder (e.g., about 10%, about 20%, about 30%, about 40%, about 50%, about 60%, about 70%, about 80%, or about 90% alcohol/water (v/v) extract of Uncaria tomentosa bark powder). Exemplary alcohols include, but are not limited to, ethanol, methanol, propanol, and butanol.

The Uncaria tomentosa extract may be filtered to remove insoluble and/or high molecular weight material and concentrated. Any conventional filtration and concentration methods known to a person of ordinary skill in the art may be used. For example, extracts of Uncaria tomentosa bark powder may be filtered through a filter paper or membrane with a pore size of 1-10 μm, 11-20 μm, 21-30 μm, 31-40 μm, 41-50 μm, 51-60 μm, 61-70 μm, 71-80 μm, 81-90 μm, 91-100 μm, or less than 1μm. Methods for concentrating the Uncaria tomentosa extract include but are not limited to evaporation, vacuum concentration, lyophilization, reverse extraction, solute precipitation, and dialysis (solvent exchange).

In some embodiments, the Uncaria tomentosa extract is derived from the inner bark and/or roots of the tree. In some embodiments, the Uncaria tomentosa extract is derived from the inner bark and/or roots from the Amazon or Brazilian rainforest.

In some embodiments, the composition comprises from about 100 mg to about 500 mg of an Uncaria tomentosa extract. In some embodiments, the composition comprises from about 100 mg to about 300 mg of an Uncaria tomentosa extract. In some embodiments, the composition comprises from about 100 mg to about 250 mg of an Uncaria tomentosa extract. In some embodiments, the composition comprises about 100 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg of an Uncaria tomentosa extract.

In some embodiments, the composition comprises from about 5% to about 60% w/w Uncaria tomentosa extract. In some embodiments, the composition comprises from about 1% to about 40% w/w Uncaria tomentosa extract. In some embodiments, the composition comprises from about 10% to about 100% w/w Uncaria tomentosa extract. In some embodiments, the composition comprises from about 10% to about 40% w/w Uncaria tomentosa extract. In some embodiments, the composition comprises about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60% about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% w/w Uncaria tomentosa extract.

For additional information and background on Uncaria tomentosa , the reader is also referred to the inventors' WIPO International publication number WO1998/051302, which is incorporated herein by reference in its entirety. It is noted that extracts from other species of Uncaria may also be used for the present disclosure.

In some embodiments, the compositions are formulated into a single dosage form, such as a capsule. In some embodiments, the capsule is from about 100 mg to about 2000 mg. In some embodiments, the capsule is from about 100 mg to about 1000 mg. In some embodiments, the capsule is from about 200 mg to about 2000 mg. In some embodiments, the capsule is from about 200 mg to about 1000 mg. In some embodiments, the capsule is about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 670 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

### Therapeutic Use of Compositions of the Present Technology

The compositions of the present technology are useful in methods for treating coronavirus infection. Without wishing to be bound by theory, it is believed that the ability of Uncaria tomentosa extracts to treat a coronavirus infection (e.g., SARS-CoV-2 infection or COVID-19) may be attributable to their potent anti-viral, anti-mutagenic and anti-inflammatory activity, as well as their ability to generally enhance the immune system.

In one aspect, the present disclosure provides a method for treating coronavirus infection in a subject in need thereof, comprising administering to the subject an effective amount of a composition of the present technology. Examples of coronavirus infection include, but are not limited to, Middle East Respiratory Syndrome (MERS), Severe Acute Respiratory Syndrome (SARS), and SARS-CoV-2 infection (e.g., COVID-19). Additionally or alternatively, in some embodiments of the methods disclosed herein, the subject is at risk for contracting a coronavirus infection, or has contracted a coronavirus infection.

In some embodiments, the coronavirus infection may be caused by any variant of a coronavirus, e.g., a MERS-Cov variant, a SARS-Cov variant, or a SARS-CoV-2 variant. In some embodiments, the SARS-CoV-2 variant may be, but not limited to, any of those listed in Table 1.

Symptoms of coronavirus infection may include one or more of coughing, dizziness, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness, confusion, blueish face or lips, coughing up blood, decreased white blood cell count, and palpitations. Coronavirus infection may cause one or more complications selected from the group consisting of sinusitis, otitis media, pneumonia, acute respiratory distress syndrome, disseminated intravascular coagulation, pericarditis, pulmonary fibrosis, viral sepsis, and kidney failure.

Additionally or alternatively, in some embodiments, administration of the composition of the present technology ameliorates or eliminates one or more of the following: lymphopenia, eosinopenia, neutrophil/lymphocyte ratio >3.13, thrombocytopenia, and decreased values of CD4+T and CD8+ lymphocytes and NK cells.

Additionally or alternatively, in some embodiments, administration of the composition of the present technology reduces the levels of one or more of inflammatory cytokines including IL-1, IL2, IL6, IL7, IL-8, IL10, IFN-γ, TNF-IL-2R, GM-CSF, IL-1 β, GCSF, IP10, MCP1, MIP1A, and TNFα in the subject.

Additionally or alternatively, in some embodiments, administration of the composition of the present technology reduces or eliminates one or more of: sparse bilateral consolidations accompanied by ground glass opacities, peripheral/subpleural images, predominantly in the lower lobes, as evidenced by plain chest X-rays; multifocal, bilateral, peripheral/subpleural ground glass opacities, generally affecting the posterior portions of the lower lobes, with or without associated consolidations as evidenced by computed tomography of the chest; B-lines, consolidations and pleural thickening, as evidenced by pulmonary ultrasonography.

Additionally or alternatively, in some embodiments, administration of the composition of the present technology reduces one or more of severe disease rate, hospitalization rate, fatality rate, duration of hospital, and recovery time of patients with coronavirus infection (e.g., SARS-Cov-2 infection or COVID-19).

In any and all embodiments of the methods disclosed herein, the composition is administered orally, topically, intranasally, systemically, intravenously, subcutaneously, intraperitoneally, intradermally, intraocularly, iontophoretically, transmucosally, intramuscularly, intrathecally, intracerebrally, intranodally, intrapleurally, intraarterially, intracapsularly, intraorbitally, transtracheally, or intracerebroventricularly.

Additionally or alternatively, in some embodiments, the methods of the present technology further comprise separately, sequentially or simultaneously administering to the subject an additional therapeutic agent. In some embodiments, the additional therapeutic agent may be an anti-inflammatory agent, an antiviral agent, a monoclonal antibody, convalescent plasm collected from subjects recovered from coronavirus infection (e.g., SARS-Cov-2 infection or COVID-19), a vitamin, an anticoagulant, or any combination thereof.

The compositions of the present technology may be separately, sequentially or simultaneously administered to the subject with coronavirus infection (e.g., SARS-Cov-2 infection or COVID-19) at least one additional therapeutic agent. Examples of additional therapeutic agents include, but are not limited to baricitinib, Vitamin C, Vitamin D, zinc, hesperetinj, melatonin, an anticoagulant, oxygen therapy, antivirals (Lopinavir, Ritonavir, Ribavirin, Favipiravir (T-705), remdesivir, oseltamivir, chloroquine, hydroxychloroquine, merimepodib, and Interferon), dexamethasone, prednisone, methylprednisolone, hydrocortisone, anti-inflammatory therapy, convalescent plasma therapy, bamlanivimab, etesevimab, casirivimab, imdevimab, and combinations thereof.

The compositions of the present technology may be used for treating coronavirus infection (e.g., SARS-Cov-2 infection or COVID-19) in a subject in need thereof. In some embodiments, the compositions of the present technology may be used for treating coronavirus infection (e.g., SARS-Cov-2 infection or COVID-19) in a human. In preferred embodiments in which the subject is a human, the subject may be at least 40 years old, at least 45 years old, at least 50 years old, at least 55 years old, at least 60 years old, at least 65 years old, at least 70 years old, at least 75 years old, or at least 80 years old or older. In some embodiments, the subject is a pediatric subject (i.e., less than 18 years old).

### Formulations

Compositions of the present technology can take the form of tablets, pills, capsules, semisolids, powders, sustained release formulations, solutions, beverages, beverage shots, suspensions, elixers, aerosals, or any other appropriate compositions, and comprise at least one pharmaceutically acceptable excipient, carrier, or diluent. Suitable excipients, carriers and diluents are well known to persons of ordinary skill in the art. The methods of formulating the compositions, can be found in standard references as Alfonso A R: Remington's Pharmaceutical Sciences, 17^{th} ed., Mack Publishing Company, Easton, Pa., 1985. Suitable liquid carriers, especially for injectable solutions, include water, aqueous saline solution, aqueous dextrose solution, and glycols.

In particular, the compound(s) can be administered orally, for example, as tablets, trouches, lozenges, aqueous or oily suspensions, dispersible powders or granules, dissolving fizz tablets, emulsions, hard or soft capsules, syrups or elixers. Compositions intended for oral use can be prepared to any method known in the art for the manufacture of nutraceutical compositions and such compositions can contain one or more agents selected from the group consisting of sweetening agents, flavoring agents, coloring agents and preserving agents in order to provide nutraceutically elegant and palatable preparations.

In some embodiments, the components of the compositions are obtained commercially in any form could be further modulated using suitable carriers, excipients and diluents including lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the present technology may be formulated so as to provide quick, sustained or delayed response of the active ingredient after administration to the subject.

Tablets containing the extracts described herein in admixture with non-toxic pharmaceutically acceptable excipients that are suitable for the manufacture of tablets. These excipients include, for example, inert diluents, such as calcium carbonate, sodium carbonate, lactose, calcium phosphate or sodium phosphate; granulating and disintegrating agents, for example, maize starch or alginic acid; binding agents, for example, maize starch, gelatin or acacia; and lubricating agents, for example magnesium stearate or stearic acid or talc. The tablets can be uncoated or they can be coated by known techniques to delay disintegration and absorption in the gastrointestinal tract and thereby provide a sustained action over a longer period. For example, a time delay material such as glycerol monostearate or glycerol distearate can be employed. Formulations for oral use can also be prepared as hard gelatin capsules wherein the compounds are mixed with an inert solid diluent, for example, calcium carbonate, calcium phosphate or kaolin, or as soft gelatin capsules, wherein the active ingredient is mixed with water or an oil medium, for example, peanut oil, liquid paraffin or olive oil.

Aqueous suspensions contain the compound in admixture with excipients suitable for the manufacture of aqueous suspensions. Such excipients include, for example, suspending agents, for example, sodium carboxymethylcellulose, methylcellulose, hydroxypropylmethyl cellulose, sodium alginate, polyvinylpyrrolidone, gum tragacanth and gum acacia; and dispersing and wetting agents that are naturally occurring phosphatides, for example lecithin, or condensation products of an alkylene oxide with fatty acids; for example polyoxyethylene stearate, or condensation products of ethylene oxide with long chain aliphatic alcohols, for example heptadecaethyleneoxycetanol, or condensation products of ethylene oxide with partial esters derived from fatty acids such as hexitol, for example polyoxyethylene sorbitol monooleate, or condensation products of ethylene oxide with partial esters from fatty acids and a hexitol annyhydride, for example polyethylene sorbitan monooleate. The aqueous suspensions can also contain one or more preservatives, for example ethyl or n-propyl p-hydroxybenzoate, one or more coloring agents, one or more flavoring agents, and/or one or more sweetening agents, such as sucrose or saccharin.

Oily suspensions can be formulated by suspending the extracts in a vegetable oil, for example arachs oil, olive oil, sesame oil, or coconut oil, or in a mineral oil such as liquid paraffin. The oily suspensions can contain a thickening agent for example beeswax, hand paraffin or cetyl alcohol. Sweetening agents, such as those set forth below, and flavoring agents can be added to provide a palatable oral preparation. These compositions can be preserved by the addition of an antioxidant such as ascorbic acid.

Dispersible powders and granules suitable for preparation of an aqueous suspension by the addition of water provide the active ingredients in admixture with a dispersing or wetting agent, a suspending agent and one or more preservatives. Suitable dispersing or wetting agents and suspending agents are exemplified by those already described above. Additional excipients, for example sweetening, flavoring and agents, can also be present.

The compositions can also be in the form of oil-in-water emulsions. The oily phase of a vegetable oil, for example, olive oil or arachis oils, or a mineral oil, for example liquid paraffin, or mixtures thereof. Suitable emulsifying agents can be naturally-occurring gums, for example gum acacia or gum tragacanth, naturally occurring phosphatides, for example soy bean, lecithin, and esters or partial esters derived from fatty acids and hexitol anhydrides, for example sorbitan esters with ethylene oxide, for example polyoxyethylene sorbitan monooleate, and condensation products of the said partial esters with ethylene oxide, for example polyethylene sorbitan monooleate. The emulsion can also contain sweetening and flavoring agents. Syrups and elixers can be formulated with sweetening agents, for example, glycerol, sorbitol or sucrose. Such formulations can also contain a demulcent, a preservative and flavoring and coloring agents.

### Modes of Administration and Effective Dosage of the Compositions of the Present Technology

Any method known to those in the art for contacting a cell, organ or tissue with a composition of the present technology may be employed. Suitable methods include in vitro, ex vivo, or in vivo methods. In vivo methods typically include the administration of a composition of the present technology, such as those described above, to a mammal, suitably a human. When used in vivo for therapy, the compositions of the present technology are administered to the subject in effective amounts (i.e., amounts that have desired therapeutic effect). The dose and dosage regimen will depend upon the degree of the infection in the subject, the characteristics of the particular composition used, e.g., its therapeutic index, the subject, and the subject's history.

The effective amount may be determined during pre-clinical trials and clinical trials by methods familiar to physicians and clinicians. An effective amount of a composition useful in the methods may be administered to a subject in need thereof by any of a number of well-known methods for administering pharmaceutical compositions. The composition may be administered systemically or locally.

The composition may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compositions that are not intended for administration to a patient. Pharmaceutically acceptable salts can be derived from pharmaceutically acceptable inorganic or organic bases and from pharmaceutically acceptable inorganic or organic acids. In addition, when a composition contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like.

The composition described herein, or a pharmaceutically acceptable salt thereof, can be incorporated into pharmaceutical compositions for administration, singly or in combination, to a subject for the treatment of coronavirus infection described herein. Such compositions typically include the active agent and a pharmaceutically acceptable carrier. As used herein, the term “pharmaceutically acceptable carrier” includes saline, solvents, dispersion media, coatings, antibacterial and antifungal agents, isotonic and absorption delaying agents, and the like, compatible with pharmaceutical administration. Supplementary active compositions can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The compositions can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compositions of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. In some embodiments, the compositions of the present technology are formulated as a pill, tablet, caplet, soft or hard gelatin capsule, lozenge, sachet, cachet, vegicap, liquid drop, elixir, suspension, emulsion, solution, beverage preparation, cold or hot tea beverage, syrup, tea bag, aerosol, suppository, sterile injectable solution, or sterile packaged powder

For administration by inhalation, the compositions can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compositions are formulated into ointments, salves, gels, or creams as generally known in the art. In one embodiment, transdermal administration may be performed by iontophoresis.

A composition can be formulated in a carrier system. The carrier can be a colloidal system. The colloidal system can be a liposome, a phospholipid bilayer vehicle. In one embodiment, the composition is encapsulated in a liposome while maintaining composition integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the composition can be embedded in the polymer matrix, while maintaining composition integrity. The polymer may be natural, such as polypeptides, proteins or polysaccharides, or synthetic, such as poly a-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compositions are prepared with carriers that will protect the therapeutic compositions against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compositions can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit high therapeutic indices are advantageous. While compositions that exhibit toxic side effects may be used, care should be taken to design a delivery system that targets such compositions to the site of affected tissue in order to minimize potential damage to uninfected cells and, thereby, reduce side effects.

The data obtained from the cell culture assays and animal studies can be used in formulating a range of dosage for use in humans. The dosage of such compositions may be within a range of circulating concentrations that include the ED₅₀ with little or no toxicity. The dosage may vary within this range depending upon the dosage form employed and the route of administration utilized. For any composition used in the methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the composition, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of the composition ranges from 0.001-10,000 micrograms per kg body weight. In one embodiment, composition concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime. In some embodiments, a therapeutically effective amount of a composition may be defined as a concentration of the composition at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The subject treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### EXAMPLES

The present technology is further illustrated by the following Examples, which should not be construed as limiting in any way.

### Example 1

### In Vitro Evaluation of Antiviral Activity of Uncaria tomentosa Extract Against Coronavirus Infection

Standard in vitro antiviral assays will be carried out to measure the effects of these Uncaria tomentosa extracts on cytotoxicity, virus yield and infection rates of coronavirus (e.g., SARS-Cov-2). Examples of these assays may be found in Wang, M., et al. Cell Res 30: 269-271 (2020); Touret, F., et al. Sci Rep 10: 13093 (2020); Aleksandr I., et al., bioRxiv 2020.09.17.299933 (2020); Ogando, N. S. et al., J. Gen Virol 101: 925-940 (2020); and Tan E. L., et al., Emerg Infect Dis 10: 581-586 (2004), which are incorporated by reference herein in their entireties.

### Cell Viability Assays

Cytotoxicity of Uncaria tomentosa extracts in human cells (e.g., Vero E6 cells (ATCC-1586)) will be determined by a cell viability assay (e.g., the CCK8 assay, or MTT assay). Take MTT assay for example. Briefly, Vero E6 will be seeded at cell density of 1.0×10⁴ cells/well in 96-well plates and incubated for 24 hours at 37° C. in a humidified 5% CO₂ atmosphere. After, 100 μL of serial dilutions (1:2) of U. tomentosa extract ranging from 3.1 to 50 μg/mL will be added to each well and incubated for 48 hours, at 37° C. with 5% CO₂. After incubation, supernatants will be removed, cells will be washed twice with Phosphate Buffered Saline (PBS) and 30 μL of the MTT reagent (2 mg/mL) will be added. The plates will be incubated for 2 hours at 37° C., with 5% CO₂, protected from light. Then, formazan crystals will be dissolved by adding 100 μL of pure DMSO to each well. Plates will be read in a multiskan GO spectrophotometer (Thermo) at 570 nm. The average absorbance of cells without treatment will be considered as 100% of viability. Based on this control the cell viability of each treated well will be calculated. The treatment concentration with 50% cytotoxicity (The 50% cytotoxic concentration—CC₅₀) will be obtained by performing nonlinear regression followed by the construction of a concentration-response curve (GraphPad Prism). For MTT assay, 2 independent experiments with four replicates each experiment will be performed (n=8).

### Antiviral Assay

Human cells (e.g., Vero E6 cells) will be infected with coronavirus (e.g., SARS-Cov-2) at a multiplicity of infection (MOI) of 0.05 in the presence of varying concentrations of the test Uncaria tomentosa extracts (e.g., PTI-00703). DMSO will be used in the control samples. Efficacies will be evaluated by quantification of viral copy numbers in the cell supernatant via quantitative real-time RT-PCR (qRT-PCR) and confirmed with visualization of virus nucleoprotein (NP) or S protein expression through methods including but not limited to immunofluorescence microscopy, ELISA, and CLIA at 48 hour post infection (p.i.) (cytopathic effect is not obvious at this time point of infection).

Provided below is a specific protocol for an in vitro antiviral assay based on visualization of coronavirus spike (S) protein expression through immunofluorescence microscopy, according to US Patent No. 10695361, which is incorporated by reference herein in its entirety, with minor adaptions. Briefly, Vero E6 cells will be seeded in 96-well plates and serial dilutions of Uncaria tomentosa extracts (e.g., PTI-00703) will be added to the assay plates by direct titration. The cells will be infected with coronavirus (e.g., SARS-Cov-2) at a multiplicity of infection of 0.5 plaque forming unit (pfu) per cell. The infected cultures will be incubated for 48 hours. The level of virus replication in Uncaria tomentosa extracts-treated and control vehicle-treated cultures will be determined by quantifying the level of virus-specific antigen following immuno-staining with antibody against coronavirus (e.g., SARS-Cov-2) spike (S) protein. A commercially available primary antibody against coronavirus spike (S) protein will be diluted 1000-fold in blocking buffer (× phosphate buffered saline (PBS) with 3% BSA) and added to each well of the assay plate. The assay plates will be incubated for 60 minutes at room temperature. The primary antibody will be removed and the cells will be washed 3 times with 1× PBS. The secondary detection antibody against the primary antibody will be conjugated with a fluorophore (e.g., Dylight488). The secondary antibody will be diluted 1000-fold in blocking buffer and added to each well in the assay plate. The assay plates will be incubated for 60 minutes at room temperature. Nuclei will be stained using Draq5 (Biostatus, Shepshed Leicestershire, UK, Cat # DR05500) diluted in 1× PBS. The cells will be counter-stained with CellMask Deep Red (Thermo Fisher Scientific, Waltham, Mass., Cat #C10046) to enhance detection of the cytoplasm compartment. Cell images will be acquired using Perkin Elmer Opera confocal microscope (Perkin Elmer, Waltham, Mass.) using 10× air objective to collect 5 images per well. Virus-specific antigen will be quantified by measuring fluorescence emission (e.g., at a 488 nm wavelength for Dylight488) and the nuclei will be quantified by measuring fluorescence emission at a 640 nm wavelength. High content image analysis will be performed to quantify the percent of infected cells and cell viability. Analysis of dose response to determine EC50 values will be performed using GeneData Screener software applying Levenberg-Marquardt algorithm for curve fitting strategy.

Alternatively, coronavirus (e.g., SARS-Cov-2) in the cell culture supernatants will be quantified by virus titration by plaque assay and TCID₅₀ assay. The supernatant of infected cells without treatment will be used as infection control. Chloroquine (CQ) at 50 μM will be used as positive control for antiviral activity; 2 independent experiments with 3 replicates of each experiment will be performed (n=6).

Plaque assay. The capacity of U. tomentosa extract to decrease the PFU/mL of coronavirus (e.g., SARS-Cov-2) will be evaluated by plaque assay on Vero E6 cells. Briefly, 1.0×10⁵ Vero E6 cells per well will be seeded in 24-well plates for 24 hours, at 37° C., with 5% CO2. Tenfold serial dilutions of the supernatants obtained from the antiviral assay (200 uL per well) will be added by duplicate on cell monolayers. After incubation during 1 h, at 37° C., with 5% CO_{2,} the viral inoculum will be removed and 1 mL of semi-solid medium (1.5% carboxymethyl-cellulose in DMEM 1× with 2% FBS and 1% Penicillin-Streptomycin) will be added to each well. Cells will be incubated for 5 days at 37° C., with 5% CO₂. Then, cells will be washed twice with PBS. After, cells will be fixed and stained with 500 uL of 4% Formaldehyde/1% Crystal violet solution for 30 minutes and washed with PBS. Plaques obtained from each condition will be counted. The reduction in the viral titer after treatment with each concentration of U. tomentosa extract compared to the infection control is expressed as inhibition percentage. Two independent experiments with two replicates of each experiment will be performed (n=4).

TCID₅₀ for SARS-CoV-2 quantification. The capacity of U. tomentosa extract to diminish the CPE caused by SARS-CoV-2 on Vero E6 will be evaluated by TCID₅₀ assay. Briefly, 1.2×10⁴ Vero E6 cells per will be seeded in 96-well plates for 24 hours, at 37° C., with 5% CO₂. Tenfold serial dilutions of the supernatants obtained from the antiviral assay (50 per well) will be added by quadruplicate on cell monolayers. After 1 hour incubation, at 37° C. with 5% CO₂, the viral inoculum will be removed and replaced by 170 μL of DMEM supplemented with 2% FBS. Cells will be incubated for 5 days at 37° C., with 5% CO₂. Then, cells will be washed twice with PBS, and then fixed and stained with 100 uL/well of 4% Formaldehyde/1% Crystal violet solution for 30 minutes. Cell monolayers will be washed with PBS. The number of wells positive for CPE will be determined for each dilution (CPE is considered positive when more that 30% of cell monolayer if compromised). The viral titer of TCID₅₀/mL will be calculated based on Spearman-Kaerber method. The reduction of viral titer after treatment with each concentration of U. tomentosa extract compared to infection control is expressed as inhibition percentage. A control of cells without infection and treatment will be included. Two independent experiments with two replicates of each experiment will be performed (n=4).

It is anticipated that Uncaria tomentosa extracts at various concentrations will reduce virus yield and inhibit infection in a dose-dependent manner. Accordingly, Uncaria tomentosa extracts are useful in methods for treating coronavirus infection in a subject in need thereof.

### Example 2

### In Vivo Evaluation of the Effects of Uncaria tomentosa Extracts on Coronavirus Infection in Animal Models

Effects of Uncaria tomentosa extracts on coronavirus infection will be examined using animal models. The animal models for coronavirus infection are known to those skilled in the art. For example, various non-primate animal models for Covid-19 including mouse, Syrian hamster model, Ferrets (Mustela putorius furo), mink (Neovison vison), domestic cat (Felis catus), dogs (Canis lupus familiaris), pig, chicken, duck, and fruit bats, and non-human-primate animal models including rhesus macaques (Macaca mulatta), cynomolgus macaques (Macaca fascicularis) and African green monkeys (Chlorocebus aethiops), have been described in Muñoz-Fontela, C., et al. Nature 586: 509-515 (2020).

Provided below are non-limiting examples of using an animal model for in vivo evaluation of the effects of Uncaria tomentosa extracts on coronavirus infection.

In vivo toxicity of Uncaria tomentosa extracts in the animal models will be determined. Animal models will be infected with coronavirus (e.g., SARS-Cov-2) and treated with Uncaria tomentosa extracts (e.g., PTI-00703® cat's claw) or PBS controls. The Uncaria tomentosa extracts (e.g., PTI-00703® cat's claw) or PBS controls will be administered prior to, simultaneous with, or subsequent to coronavirus infection. Efficacy will be evaluated by monitoring lung function, viral load, and/or pathological examination. For example, efficacy may be evaluated using a mouse model for coronavirus infection, according to the methods described in U.S. Pat. No. 10,695,361, which is incorporated by reference herein in its entirety.

Changes in lung function will be determined by whole body plethysmography (WBP, Buxco lung function testing system, Data Sciences International). After a 30-minute acclimation in the plethysmograph chamber, 11 respiratory responses and several quality control metrics will be continually measured every 2-second for 5 minutes for a total of 150 data points. Mean values for each parameter will be determined within DSI Finepoint software.

Histological analysis will be performed on formalin fixed samples and paraffin embedded tissues with 5 μm. To assess lung pathology, sections will be stained with hematoxylin and eosin. Viral antigen in the lung will be stained using monoclonal or polyclonal antibody against a viral antigen (e.g., nucleocapsid or S protein). Slides will be blinded to the evaluator and assessed for virus associated lung pathology as well as spatial location and prevalence of viral antigen. Images are captured using an Olympus BX41 microscope equipped with an Olympus DP71 camera.

Viral plaque assay will be used to quantify infectious virus from frozen lung tissue. Vero E6 cells will be seeded in 6-well plates at 5×10⁵ cells/well. Lung tissue will be thawed, and homogenized via Roche Magnalyzer. The tissue suspension will be serially diluted and the dilutions will be used to infect the Vero E6 cells. At 72 hour post-infection, the plates will be fixed and stained and the number of plaques quantified by visual inspection. The primary endpoint for this study is viral load in lung tissue at Day 5 post-infection.

Additional endpoints will include changes in animal lung function, and/or body weight rate. Animal body weight will be recorded daily for the duration of the in-life phase. On day −1, 1, 2, 3, and 5 after inoculation, whole body plethysmography will be performed to assess lung function. On Day 5, a scheduled necropsy will be performed on all remaining animals; gross lung pathology is evaluated by a board-certified veterinary pathologist. Lung tissue will be collected for histopathological and virological analysis.

It is anticipated that, compared to untreated controls, animals treated with Uncaria tomentosa extracts will show better lung function, decreased infectious virus, and/or higher body weight at Day 2 and/or Day 5 post-infection. Accordingly, Uncaria tomentosa extracts are useful in methods for treating coronavirus infection in a subject in need thereof.

### Example 3

### Clinical Trials for Evaluation of Uncaria tomentosa Extracts to Treat a Coronavirus Infection

Subjects with coronavirus infection (e.g., COVID-19) will be evaluated to determine clinical status prior to peroral or systemic administration of Uncaria tomentosa extracts, or a control vehicle. The Uncaria tomentosa extract, or the control vehicle will be administered to each subject according to the intended route of administration. Clinical monitoring is conducted to determine therapeutic response.

It is anticipated that, compared to untreated controls, subjects treated with Uncaria tomentosa extracts will show reduction of one or more of coronavirus infection (e.g., COVID-19) symptoms including but not limited to fever, dizziness, cough, shortness of breath, fatigue, headache, loss of taste or smell, nasal congestion, sore throat, coughing up sputum, pain in muscles or joints, chills, nausea, vomiting, and diarrhea, difficulty waking, confusion, blueish face or lips, coughing up blood, decreased white blood cell count, and kidney failure, and/or amelioration of one or more of coronavirus infection (e.g., COVID-19) complications including but not limited to pneumonia, pulmonary fibrosis, viral sepsis, acute respiratory distress syndrome, and kidney failure, and/or reduction of one or more of severe disease rate, hospitalization rate, fatality rate, duration of hospital, and recovery time of patients with coronavirus infection (e.g., COVID-19).

Additionally or alternatively, it is anticipated that, compared to untreated controls, subjects treated with Uncaria tomentosa extracts will ameliorate or eliminate one or more of the following: lymphopenia, eosinopenia, neutrophil/lymphocyte ratio >3.13, thrombocytopenia, decreased levels of CD4+T and CD8+ lymphocytes and NK cells, and increased levels of inflammatory cytokines including IL-1, IL2, IL6, IL7, IL-8, IL10, IFN-γ, TNF-IL-2R, GM-CSF, and IL-1 β, GCSF, IP10, MCP1, MIP1A, and TNFα.

Additionally or alternatively, it is anticipated that, compared to untreated controls, subjects treated with Uncaria tomentosa extracts will reduce or eliminate one or more of: sparse bilateral consolidations accompanied by ground glass opacities, peripheral/subpleural images, predominantly in the lower lobes, as evidenced by plain chest X-rays; multifocal, bilateral, peripheral/subpleural ground glass opacities, generally affecting the posterior portions of the lower lobes, with or without associated consolidations as evidenced by computed tomography of the chest; B-lines, consolidations and pleural thickening, as evidenced by pulmonary ultrasonography.

Accordingly, Uncaria tomentosa extracts are useful in methods for treating coronavirus infection in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating coronavirus infection in a subject in need thereof comprising administering to the subject a composition comprising a therapeutically effective amount of an Uncaria tomentosa extract.

2. The method of claim 1, wherein the composition comprises from about 100 mg to about 500 mg of an Uncaria tomentosa extract.

3. The composition of claim 1, wherein the composition comprises from about 10% to about 40% w/w Uncaria tomentosa extract.

4. The method of claim 1, wherein the composition is formulated as a pill, tablet, caplet, soft or hard gelatin capsule, lozenge, sachet, cachet, vegicap, liquid drop, elixir, suspension, emulsion, solution, beverage preparation, cold or hot tea beverage, syrup, tea bag, aerosol, suppository, sterile injectable solution, or sterile packaged powder.

5. The method of claim 1, wherein the composition is formulated as a capsule.

6. The method of claim 5, wherein the capsule is from about 200 mg to about 1000 mg.

7. The method of claim 1, wherein the subject is at risk for contracting a coronavirus infection.

8. The method of claim 1, wherein the subject has contracted a coronavirus infection.

9. The method of claim 1, wherein the coronavirus infection is selected from the group consisting of Middle East Respiratory Syndrome (MERS), Severe Acute Respiratory Syndrome (SARS), SARS-CoV-2 infection, and COVID-19.

10. The method of claim 9, wherein the coronavirus infection is SARS-CoV-2 infection or COVID-19.

11. The method of claim 1, wherein the coronavirus infection comprises one or more symptoms selected from the group consisting of coughing, dizziness, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness, confusion, blueish face or lips, coughing up blood, decreased white blood cell count, and palpitations.

12. The method of claim 1, wherein the coronavirus infection causes one or more complications selected from the group consisting of sinusitis, otitis media, pneumonia, acute respiratory distress syndrome, disseminated intravascular coagulation, pericarditis, pulmonary fibrosis, viral sepsis, and kidney failure.

13. The method of claim 1, wherein the composition is administered orally, topically, intranasally, systemically, intravenously, subcutaneously, intraperitoneally, intradermally, intraocularly, iontophoretically, transmucosally, intramuscularly, intrathecally, intracerebrally, intranodally, intrapleurally, or intracerebroventricularly.

14. The method of claim 1, further comprising separately, sequentially or simultaneously administering one or more additional therapeutic agents to the subject.

15. The method of claim 1, wherein the one or more additional therapeutic agents are selected from among baricitinib, Vitamin C, Vitamin D, zinc, hesperetinj, melatonin, an anticoagulant, oxygen therapy, antivirals (Lopinavir, Ritonavir, Ribavirin, Favipiravir (T-705), remdesivir, oseltamivir, chloroquine, hydroxychloroquine, merimepodib, and Interferon), dexamethasone, prednisone, methylprednisolone, hydrocortisone, anti-inflammatory therapy, convalescent plasma therapy, bamlanivimab, etesevimab, casirivimab, imdevimab, and combinations thereof.
